SEQUENCE LISTING (6 sequences)

SEQ ID NO: 1 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 2 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 3 - protein, 365 aa, Human papillomavirus type 16
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 4 - protein, 105 aa, Human papillomavirus type 18
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 5 - protein, 158 aa, Human papillomavirus type 18
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 6 - protein, 365 aa, Human papillomavirus type 18
MQTPKETLSERLSCVQDKIIDHYENDSKDIDSQIQYWQLIRWENAIFFAAREHGIQTLNHQVVPAYNISKSKAHKAIELQMALQGLAQSRYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTM

(19) United States
(12) Patent Application Publication
(10) Pub. No.: US 2009/0028874 A1
(43) Pub. Date: Jan. 29, 2009

(54) Synthetic Protein as Tumor-Specific Vaccine

(75) Inventors: Sjoerd Henricus Van Der Burg, Waddinxveen (NL); Jan Wouter Drijfhout, Leiden (NL)

Correspondence Address: THE WEBB LAW FIRM, P.C., 700 KOPPERS BUILDING, 436 SEVENTH AVENUE, PITTSBURGH, PA, 15219, US

(73) Assignee: LEIDEN UNIVERSITY MEDICAL CENTER, Leiden (NL)

(21) Appl. No.: 10/583,837

(22) PCT Filed: Dec. 24, 2003
(86) PCT No.: PCT/NL2003/000929

§ 371 Date: Jun. 22, 2006

(51) Int. Cl. A61K 39/395; C07K 1/00; A61K 39/00; A61P 31/00; A61K 38/00

(52) U.S. Cl. 424/172.1; 530/333; 514/12; 424/186.1

(57) ABSTRACT

The invention provides a GMP compatible method to chemically synthesize proteins which may be advantageously used in compositions for vaccination that are free of biological contaminants. The method uses conventional synthesis of peptides and linking these to yield synthetic proteins that preferably comprise all T cell epitopes for an antigen. Preferably an adjuvant is covalently attached to a synthetic protein to yield a fully synthetic vaccine. The invention is illustrated mainly by using HPV protein directed immunity as a model.

### FIELD OF THE INVENTION

The present invention relates generally to the field of medicine, and more specifically to induction and/or enhancement of a T cell response directed towards an antigen, using synthetic peptides and linking these to yield synthetic proteins that comprise all T cell epitopes for said antigen. Preferably an adjuvant is covalently attached to a synthetic protein to yield a synthetic vaccine. The invention is exemplified mainly by using HPV directed immunity as a model. However, the invention should not be read as being limited to HPV but rather as being relevant for a wide variety of immune related or relatable diseases.

### BACKGROUND OF THE INVENTION

HPV infection is highly prevalent among young, sexually active male and female individuals. Large prospective studies showed that acquisition of HPV from male partners is common, occurring in 40-60% of subjects during a 3 year follow-up period (Koutsky et al., Am J Med 102 (5a) 3, 1997, Ho et al., N Eng J Med 338 (7) 1998, Marrazzo et al., Am J Obstet Gynecol 183(3), 2000). Therefore, HPV is probably the most common sexually transmitted disease. Papillomaviruses of the high-risk types (e. g. HPV-16, -18, -31, -33 and -45) are responsible for cervical cancer (Bosch et al., Natl Cancer Inst 87, 1995, Zur Hausen, Bioch Biophys Acta 1288, F55 1996). Following infection of the basal epithelial cells, the immediate HPV early genes E1, E2, E6 and E7 are expressed. Prolonged and elevated expression of the E6 and E7 oncoproteins is tightly associated with HPV-induced dysplasia and transformation into cervical carcinoma.

The protective role of the immune system in the defense against HPV-related diseases and HPV-induced cancer in humans is suggested by the fact that compared to normal controls, immunosuppressed renal transplant patients and patients infected with HV display a 17-fold greater incidence of genital HPV infection (Matorras et al. Am J Obstet Gynecol 164, 42, 1991, Halpert et al., Obstet Gynecol 68(2) 1986). The diminished capacity of immunosuppressed individuals to resolve HPV infection indirectly points towards a protective role of the immune system early in infection. A positive role for HPV-specific Th-immunity was suggested by the predomination of CD4+ T-cells in regressing genital warts (Coleman et al., J. Clin. Pathol., 102, 1994) as well as by the detection of delayed-type hypersensitivity responses to HPV16E7 in the majority of subjects with spontaneous regressing CIN lesions (Hopfl et al., 18th int. Papillomavirus conference, pp 2000). Studies on HPV16-specific T-cell immunity in humans suggested that protective HPV16-specific immunity is characterized by a strong IFN-gamma and interleukin-5 associated Th1/Th2 type memory T helper-cell response reactive against the three HPV16 early proteins E2, E6 and E7 (de Jong et al., Cancer Res. 62, p 472-9, 2002; Welters et al., Cancer Res. 63, p 636-41, 2003). The majority of patients with HPV16-positive pre-malignant lesions or cervical carcinoma fail to induce this type of T-cell reactivity and the lack of T-cell help may explain the general absence of HPV16-specific CD8+ cytotoxic T lymphocytes in these patients (Ressing et al., Cancer Res. 56, 1996; Nimako et al., Cancer Res. 57, 1997). Importantly, studies on spontaneously induced protective immunity against papilloma viruses in beagles and rabbits corroborate our findings in that E2 and E6-specific T-cell responses peaked during the regression of virus-induced lesions (Moore RA, Virology 2003). Furthermore, the use of DNA-vaccines encoding the early antigens E2, E6 and E7 prevented persistent infection and associated epithelial dysplasia in these animal models (Han et al. 1999, Selvakumar et al., J Virol 69(1) 1995). These data indicate that immunity against E2, E6, and E7 can be effective as immunoprophylaxis of papillomavirus infection as well as therapeutically for HPV-induced lesions and cancer.

New insights in the molecular and cellular events leading to a succesful attack of chronic viral infections or virus-induced tumors have emerged only recently. Full mobilization of lytic effector cells crucially depends on proper activation (maturation) of DC either by innate immunity triggers such as microbial ligands of Toll like receptors (TLR) and/or by triggers of adaptive immunity such as CD40 ligand (CD40L) on activated CD4+ T helper (Th) cells (Melief et al., Immunol Rev. 188, 2002). Because infection of epithelia by HPV16 is, at least initially, not accompanied with gross disturbance of this tissue and/or strong pro-inflammatory stimuli, a successful immune response is likely to depend on HPV16-specific CD4+ T-cell help. Rather than relying on poorly defined immune system triggers such as recombinant vectors and adjuvants without molecularly defined function, it is now possible to design entirely synthetic vaccines that provide both the proper antigenic and accessory signals for induction of a full scale burst of effector T-cells as well as T-cell memory. The main hallmark of such vaccines is that they provide not only antigens that stimulate both CD4+ and CD8+ T cells but also contain compounds that closely mimic the most successful natural triggers of dendritic cell (DC) activation (Melief et al., Immunol. Rev. 188, 2002; Zwaveling J. Immunol. 169, 2002). For a clinically relevant approach of immunizing subjects, for instance against (Myco) bacterially and/or virally infected cells or tumor cells, and HPV in particular, it is preferred that both specific T-helper cells and CTL's are induced. We have already shown that immunization with minimal CTL epitopes results in protection against tumors in some models (Feltkamp et al. Eur. J. Immunol. 23, 1993, Kast et al. PNAS, 88(6) 1991) whereas, in other models, it can lead to tolerance or functional deletion of virus-and tumor-specific CTL that when otherwise induced are protective (Toes et al. PNAS 93(15) 1996, Toes et al., J Immunol 156(10), 1996). The occurrence of tolerance or functional deletion decreases the effects of vaccination significantly. Epitopes involved with this effect were therefore not suitable for immunization purposes. Processing of exogenous antigens for presentation by MHC class I molecules by cross-priming as well as by other mechanisms is now widely recognized second pathway of processing for presentation by MHC class I, next to the well known endogenous route (Jondal et al., immunity 5(4) 1996, Reimann et al. Curr. Opin Immunol 9(4), 1997). The normal outcome of antigen processing via this pathway is CTL tolerance, unless APC activation by CD4+ T-cells takes place (Kurts et al., J Exp Med 186(12), 1997). Furthermore, in several studies with murine virus infections, a positive correlation was detected between the frequency of CTL precursors and protective immunity (Sedlik et al. J Virol 74(13) 2000, Fu et al., J Immunol 162(7) 1999). For an optimal induction of CTL, presentation of CTL epitopes preferably takes place at the surface of professional antigen presenting cells (APC's) such as dendritic cells (Mellman et al., Trends Cell Biol 8, 1998, Rodriguez et al. Nat Cell Biol 1, 1999). Whereas minimal CTL-epitopes can, without the need of processing by professional antigen presenting cells, be presented to T-cells by any somatic cell, proteins need to be taken up and processed by professional antigen presenting cell for an optimal presentation of CTL and Th-epitopes in MHC class I and MHC class II, respectively, can occur (Manca et al. J Acquir Immune Defic Syndr 7, 1994).

The use of proteins from biological sources, either natural sources or recombinant proteins expressed in a host system, allows for the use of both small and larger proteins, either in purified form or as crude preps for vaccination purposes and is routinely applied in the art of vaccination. However, the use of proteins or recombinant proteins from biological sources requires extensive purification and quality control. Inherently the production of proteins or recombinant proteins from biological sources is subject to biological variations, various contaminants and errors. Because of the inherent variability and unpredictability of biological sources, the high rate of mutations and epigenetic changes in cell lines, bacteria, viruses and vectors used, the threat of contamination with DNA, in particular viral or recombinant DNA, the safety and quality control requirements set by regulatory authorities such as the EMEA (European Medicines Evaluation Agency), the US FDA (Food and Drug Administration) or the Japanese Pharmaceutical and Food Safety Bureau of Ministry of Health, Labour and Welfare are extensive and extremely strict. Clinical validation and approval of preparations for vaccination by the medical authorities and mandatory use of GMP grade materials, equipment and procedures make the use of proteins and recombinant proteins from biological sources extremely laborious, risky, costly and generally unattractive.

WO 02/070006 demonstrates the use of medium-sized synthetic peptides of 22-45 amino acids, which combine CTL and T-helper epitopes and are sufficiently large to be taken up by a professional antigen presenting cell (APC). A problem associated with the use of small or medium sized synthetic peptides is their short half-life in vivo and rapid clearing from the bloodstream, limiting the overall effectiveness of the composition for vaccination or vaccine. Furthermore the short stretch of amino acids limits the number of available epitopes contained within the synthetic peptide. The use of a substantially larger, or even a full size protein, containing most or all available epitopes of a given protein for all HLA molecules of the subject to be immunized, would be highly preferable. The current state of the art permits the synthesis of synthetic peptides up to roughly 60 amino acids, depending on the amino acid content. Although much longer peptides can in theory be synthesized, yield and quality progressively reduce with increasing size of the peptide.

On the one hand, the use of a highly defined, tightly controlled and monitored chemical process to obtain synthetic proteins would circumvent the problems associated with the use of proteins from biological sources. On the other hand however, the length-limitation on chemically synthesized peptides also limits their immunological effectiveness as vaccines. It is an object of the present invention to overcome these shortcomings and to provide for immunologically effective proteinaceous vaccines that are produced by chemical synthesis.

### DETAILED DESCRIPTION OF THE INVENTION

### Definitions

A synthetic or artificial peptide is defined herein as a chemically produced polymer of amino acids or polypeptide, produced by chemical synthesis of a polymer of the 20 naturally occurring amino acids linked via peptide bonds. A synthetic protein is defined as a fusion product of at least two or more synthetic peptides, ranging in length from 2 to 80 amino acids, that have been chemically synthesized. Ligation of two or more synthetic peptides yields a synthetic protein which may correspond to a part or the full length a naturally occurring protein and which may vary in length from approximately 80 amino acids to approximately 1000 amino acids, preferably from 85 to 400 amino acids and more preferably from 90 to 200 amino acids. In contrast, a natural or recombinant protein is defined as an enzymatically produced protein or polypeptide, enzymatically produced in vivo or in vitro by translation of a coding RNA template.

GMP-grade peptides are produced under Good Manufacturing Practice protocols wherein all steps in the procedure are standardized, fully documented and constantly monitored. The documentation system leads to batch records that are logical and easy to follow for auditing by authorities such as the FDA or EMEA and will facilitate quality control and monitoring required for approval and clinical use of the artificial protein.

GMP-grade proteins may be extensively tested before clinical use in a vaccine by the following non extensive list of techniques: Mass Spectrometric Analysis, Amino Acid Analysis (AAA), Peptide Sequencing, Reverse Phase-HPLC, Residual Organic Volatiles, Bacterial Endotoxin Evaluation, Bioburden Evaluation, Net Peptide Content by AAA, Counter Ion Content (such as acetate, trifluoroacetate, hydrochloride, etc.), Secondary Counter Ion Content, Water Content, Mass Balance Calculation and additional tests may include Specific Rotation (identity), Capillary Zone Electrophoresis (purity), Titrations and other chemical and biological analysis techniques obvious to the skilled person.

Adjuvants may be added to the composition to enhance and stimulate an immune response to the synthetic proteins in the preparation for vaccination. The use of various adjuvants for vaccination purposes are known in the art, (e.g. Melief Immunol Rev 188, 2002; Zwaveling J. Immunol. 169, 2002). Particularly preferred adjuvants to be used with the synthetic proteins of the current invention are bacterial LPS, CpG DNA and other Toll-like receptor activating adjuvants and dendritic cell stimulating adjuvants. In a more preferred embodiment of the invention the adjuvant used is recognized by a Toll-like-receptor (TLR) present on a professional antigen presenting cell. Various adjuvants recognized by TLR's are known in the art and include e.g. lipopeptides, lipopolysaccharides, peptidoglycans, liopteichoic acids, lipoproteins (from mycoplasma, mycobacteria or spirochetes), double-stranded RNA (poly I:C), unmethylated DNA, lipoarabinomannan, flagellin, CpG-containing DNA, and imidazoquinolines. Adjuvants may be administered with the antigen or physically attached to the antigen by chemical modification, synthesis, conjugation and other methods known in the art.

The synthetic proteins of the current invention may exhibit some sequence divergence from their naturally occurring counterparts. The term “sequence identity” means that two polypeptide sequences are identical (i.e., on an amino acid-by-amino acid basis) over a window of comparison. The term “percentage of sequence identity” is calculated by comparing two optimally aligned sequences over the window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

As applied to the peptides of the invention, the term “substantial identity” means that two peptide sequences, when optimally aligned, such as by the programs GAP or BESTFIT using default parameters, share at least 80 percent sequence identity, preferably at least 90 percent sequence identity, more preferably at least 95 percent sequence identity or more (e.g., 99 percent sequence identity). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992).

Preferably, residue positions, which are not identical, differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine.

Alignment and comparison of relatively short amino acid sequences (less than about 30 residues) is typically straightforward. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, by computerised implementations of these algorithms (GAP, BESTFIT, FASTA, and TFASTA in the Wisconsin Genetics Software Package Version 10.2, Genetics Computer Group, 575 Science Dr., Madison, Wis. 53711, USA), or by inspection, and the best alignment (i.e., resulting in the highest percentage of sequence similarity over the comparison window) generated by the various methods is selected.

### DETAILED DESCRIPTION OF THE INVENTION

In a first aspect the present invention therefore relates to a method for producing a synthetic protein. The synthetic protein preferably comprises an amino acid sequence that is at least 80, 85, 90, 95, 97, 98, 99 or 100% identical to at least 46 contiguous amino acids of a naturally occurring antigenic protein of a pathogen or tumor. The method of the invention comprises the steps of: (a) chemically synthesizing two or more fragments each consisting of 2-80 contiguous amino acids of the amino acid sequence, whereby in the amino acid sequence the two or more fragments are neighbouring and non-overlapping; (b) chemically ligating the C-terminus of a fragment to the N-terminus of a neighbouring fragment to produce the synthetic protein or a part thereof; (c) optionally, repeating step C to sequentially ligate a further neighbouring fragment obtained from step C or step B to produce the synthetic protein.

Preferably, the synthetic protein comprises an amino acid sequence that is at least 80, 85, 90, 95, 97, 98, 99 or 100% identical to at least 46, 47, 48, 50, 55, 60, 70 or 80 contiguous amino acids of a naturally occurring antigenic protein of a pathogen or tumor. More preferably, the synthetic protein comprises amino acid sequence that is at least 80, 85, 90, 95, 97, 98, 99 or 100% identical to the entire (i.e. full-length) amino acid sequence of the naturally occurring antigenic protein of a pathogen or tumor. A preferred naturally occurring antigenic protein is an HPV protein, more preferably an HPV E2, E6 or E7 protein and even more preferably the naturally occurring HPV protein is an HPV16, -18, -31, -33 or -45 protein, of which HPV16 and HPV18 proteins are most preferred. Thus, the synthetic proteins employed in the subject invention need not be identical, but may be substantially identical, to the naturally occurring antigenic protein of a pathogen or tumor. As such the synthetic proteins may be subject to various changes, such as insertions, deletions, and substitutions, either conservative or non-conservative, where such changes might provide for certain advantages in their use. Likewise, the HPV-derived synthetic proteins of the invention can be modified in a number of ways whereby the proteins preferably comprise a sequence substantially identical (as defined above) to an amino acid sequence of at least one of the HPV-derived antigenic sequences selected from SEQ ID NO's 1-6, while maintaining at least 80, 85, 90, 95, 97, 98, 99 or 100% sequence identity with one of SEQ ID NO's 1-6.

In the method of the invention, larger synthetic proteins may be synthesized by the sequential ligation of synthetic peptides as disclosed in this application. Preferably in this method in a first step a synthetic peptide is immobilized on a substrate. In subsequent steps a second and optionally more synthetic peptides are covalently attached to the peptide or fused peptides on the support by chemical means. In the examples a similar strategy is followed for the chemical synthesis of other highly immunogenic HPV proteins. The E6 proteins of HPV16 and HPV18 can be conveniently synthesized from 4 artificial peptides (example numbers 2 and 5). For the larger E2 protein of HPV16 and HPV18 two different strategies are provided by the current invention as a non-limiting example to illustrate the flexibility of the method (example numbers 3 and 6). Choosing suitable alternatives to ensure efficient linkage of individual peptides to obtain an artificial protein can circumvent sequence specific difficulties.

In a preferred embodiment the neighbouring non-overlapping fragments (i.e. the individual synthetic peptides that are used as building blocks for the synthesis of the synthetic protein by chemical ligation) are selected to comprise a N-terminal cysteine or glycine residue. The individual neighbouring non-overlapping fragments to be synthesized are preferably of moderate length (from 20 to 80 amino acids, preferably up to about 65 amino acids, more preferably up to 55 amino acids in lenght, more preferably up to 45 amino acids in lenght, most preferably up to 35 amino acids in lenght), and are preferably synthesized as their thio-ester by normal solid phase peptide synthesis, e.g. by Fmoc-based or Boc-based chemistry, on a normal solid support, e.g. on polystyrene based or polystyrene/polyethyleenglycol-copolymer based material containing a proper handle, e.g. a safety catch handle, in acceptable purity, e.g. preferably more than 80% pure. Preferably no building blocks with carboxy-terminal proline (P) are used since coupling to proline thioesters is cumbersome. In case the protein to be synthesized does not contain naturally occuring cysteines that are compatible with the preferred embodiment, e.g. because cysteins are to far apart in the sequence, it is preferred to apply a modified ligation strategy in which one or more building blocks are used with an amino-terminal glycine (G). The N-terminal G can than be modified with an protection/activation group like the N-2,3,4-trimethoxy-5-mercaptophenyl group which provides the glycine a reactivity towards thio esters that is comparable with that of a cystein and still yields a glycin in the endproduct (J. Am. Chem. Soc. 124,4642 (2002)).

The current invention is also highly suited for the production of highly toxic or unstable proteins. The described method may offer an alternative for the production of proteins, which are currently difficult to produce by recombinant technology due their inherent toxicity (e.g. HPV16 E2, wild-type p53) or their instability in vivo in the bacterial/viral/eukaryote expression systems.

In vivo injected proteins are often rapidly degraded by exopeptidases present in the extracellular matrix. In a preferrred embodiment of the current invention such degradation can be overcome by protein modification like the introduction of D-amino acids at the N- and C-termini and at strategic sites within the protein sequence. In addition proteins can be made more stable towards enzymatic degration by introduction of non-natural amino acids at particular sites within the sequence. For instance non-natural amino acids can be various forms of synthetic α-amino acids, synthetic β amino acids and/or N-methylated synthetic amino acids, features that cannot be easily achieved by conventional in vivo methods for production of proteins.

The remarkably enhanced immunogenic activity of the synthetic E7 protein compared to minimal (9 AA) or longer (35 AA) synthetic peptides is also at least in part due to the enhanced stability in vivo of a synthetic protein. This is illustrated by the fact that in contrast to synthetic E7 protein, vaccination with an equimolar dose of long E7 peptide was not efficient in the induction of CD8+ IFNγ-producing HPV16 E7-specific T-cells and this was reflected in the lack of protection against a tumour challenge. However, vaccination with a ˜8 fold higher dose of long peptide did result in a strong E7-specific T-cell response and tumour protection. The use of larger or full length (as compared to the naturally expressed counterpart of a protein) synthetic proteins or the use of specific domains, chemical modifications, removal of targeting signals for degradation from synthetic proteins, are technical measures which will help to design synthetic proteins that exhibit an enhanced half-life in vivo. Synthetic proteins with an enhanced stability are more potent inducers of an immunogenic response. The enhanced immunogenic activity, i.e. more effective in prophylactic and therapeutic tests in mice on an equimolar basis of the synthetic protein vaccine as compared to short synthetic peptides, is at least partly explained by a less rapid degradation of larger proteins and a decreased or delayed clearing from the bloodstream. After partial degradation and partial proteolysis, the remaining fragments of the larger synthetic protein may still be sufficiently large to be taken up by professional antigen presenting cells and elicit a vigorous immune response.

The (poly)peptides that are chemically ligated to form the synthetic proteins of the invention may be modified to provide a variety of desired attributes, e.g., improved pharmacological characteristics, while increasing or at least retaining substantially all of the biological activity of the unmodified peptide. For instance, the peptides can be modified by attachment of adjuvants, enhancing the stability or aid in the fusion process of synthetic peptides, enhance immunological properties by altering, extending, decreasing the amino acid sequence of the protein. Substitutions with different amino acids or amino acid mimetics can also be made.

The individual residues of the immunogenic protein derived synthetic protein of the invention can be incorporated in the synthetic protein by a peptide bond or peptide bond mimetic. A peptide bond mimetic of the invention includes peptide backbone modifications well known to those skilled in the art. Such modifications include modifications of the amide nitrogen, the a-carbon, amide carbonyl, complete replacement of the amide bond, extensions, deletions or backbone cross-links. See, generally, Spatola, Chemistry and Biochemistry of Amino Acids, Peptides and Proteins, Vol. VII (Weinstein ed., 1983). Several peptide backbone modifications are known, these include, ψ [CH₂S], ψ [CH₂NH], ψ [CSNH₂], ψ [NHCO], ψ [COCH₂ ] and ψ [(E) or (Z) CH═CH]. The nomenclature used above, follows that suggested by Spatola, above. In this context, ψ indicates the absence of an amide bond. The structure that replaces the amide group is specified within the brackets.

Amino acid mimetics may be incorporated in the synthetic proteins. An “amino acid mimetic” as used here is a moiety other than a naturally occurring amino acid that conformationally and functionally serves as a substitute for an amino acid in a peptide of the present invention. Such a moiety serves as a substitute for an amino acid residue if it does not interfere with the ability of the peptide to elicit an immune response against the relevant epiotopes, such as e.g. an HPV-derived epitope. Amino acid mimetics may include non-protein amino acids, such as β-, γ-, δ-amino acids, β-, γ-, δ-imino acids (such as piperidine-4-carboxylic acid) as well as many derivatives of L-α-amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

In another preferred embodiment of the method of the invention, the synthetic protein is chemically conjugated to an adjuvant. A preferred adjuvant is a chemically synthesized adjuvant. The conjugation of (synthetic) adjuvants to antigens is known in the art (Shirota et al., 2000. J. Immunol. 164:5575, Cho, H. J., K., et al, 2000. Nat. Biotechnol. 18:509, Tighe, H., K. et al., 2000. J. Allergy Clin. Immunol. 106:124. Tighe, H., K. et al., 2000. Eur. J. Immunol. 30:1939.)

It is also known in the art mentioned above that conjugation of antigens to adjuvants that are able to specifically activate professional antigen presenting cells such as e.g. dendritic cells via e.g. TOLL-like receptors, are superior vaccines compared to those in which both components are mixed together. In one embodiment adjuvants, preferably synthetic adjuvants, are conjugated to the synthetic proteins of the current invention, resulting in a purely synthetic and thereby completely chemically defined composition for vaccination purposes.

One suitable technology for conjugating biomolecules is known in the art as “click chemistry”. In this technology an azide moiety in one molecule is reacted with an alkyne group in another molecule in order to produce a conjugate wherein both molecules are covalently connected via a 1,2,3-triazole ring. The reaction is orthogonal to virtually all reactive groups normally present in biomolecules, is catalyzed by Cu(I) ions and can be performed in aqueous media at relative moderate reaction conditions; references Kolb H. C. et al., Angew. Chem. Int. Ed. 40, 2004-2021 (2003) and Wang, Q. et al., J. Am. Chem. Soc. 125, 3192-3193 (2003). However, other means for conjugating biomolecules and that are applicable to the antigens and adjuvants of the present invention are well known in the art.

Preferred adjuvants for combination with and/or conjugation to the antigens of the present invention are adjuvants that stimulate professional antigen presenting cells, such as dendritic cells. Such adjuvants preferably are recognized by Toll Like Receptors (TLR's) and are known in the art to include e.g. lipopeptides, lipopolysaccharides, peptidoglycans, liopteichoic acids, lipoproteins (from mycoplasma, mycobacteria or spirochetes), double-stranded RNA (poly I:C), unmethylated DNA, lipoarabinomannan, flagellin, CpG-containing DNA, and imidazoquinolines.

In one preferred embodiment of the current invention, CpG DNA is used as a TLR activating adjuvant and covalently attached to the N and/or C termini or to specific amino acids of a synthetic protein. CpG DNA and analogues thereof are known to be potent activators of TLR9. The method of conjugation of CpG to proteins is described extensively in the references (Shirota et al., 2000. J. Immunol. 164:5575, Cho, H. J., K., et al, 2000. Nat. Biotechnol. 18:509, Tighe, H., K. et al., 2000. J. Allergy Clin. Immunol. 106:124. Tighe, H., K. et al., 2000. Eur. J. Immunol. 30:1939.). A nucleophilic group like a thiol group in one of the molecules can be reacted with an electrophilic group like a haloacetyl group or a maleimido group in the other molecule. Alternatively an amine group in one of the molecules can be reacted with an activated carboxylic acid group, like a hydroxysuccinimide ester, in the other molecule. An amine group in one of the molecules can also be reacted with an amine group in the other molecule by reaction with a bifunctional crosslinker like gluteraldehyde. (Drijfhout, J. W. and Hoogerhout, P in W. C. Chan and P. D. White, Chapter 10. Oxford University Press 2000).

Also polyIC, acting upon TLR3, may be covalently attached to the synthetic proteins of the current invention. PolyIC (polyinosinic:polycytidylic acid) is a “mimic” of double-stranded viral RNA and induces interferon. As such it can be used as adjuvant in vaccination (Monshouwer M. et al., Biochem. Pharmacol. 52, 1195-1200 (1996) and Moriyama H, et al., Proc. Natl. Acad. Sci. USA 99, 5539-5544 (2002). PolyIC may also be covalently attached to a synthetic protein via the methods described in references (Shirota et al., 2000. J. Immunol. 164:5575, Cho, H. J., K., et al, 2000. Nat. Biotechnol. 18:509, Tighe, H., K. et al., 2000. J. Allergy Clin. Immunol. 106:124. Tighe, H., K. et al., 2000. Eur. J. Immunol. 30:1939.).

Imiquimod and analogues, acting upon TLR7 and 8, and Pam3Cys, acting upon TLR2, may also be advantageously used, alone or in combination with other synthetic adjuvants for conjugations to the synthetic proteins according to the current invention. Pam3Cys is a compound in which 3 palmitic acid units are bound to a cystein via a glycerol molecule and is a potent adjuvant (Rabanal, F., et al., J. Chem. Soc, Perkin Trans. 1, 945-952 (1991) and Metzger J. W. et al., Biochim. Biophys. Acta, 1149, 29-39 (1993).

In a further preferred embodiment the method comprises the step of formulating the synthetic protein into a pharmaceutical composition by mixing the protein with a pharmaceutically acceptable carrier as defined below. In one aspect the invention thus also relates to a method for producing a pharmaceutical composition comprising the synthetic proteins of the invention. The method comprises at least the steps of mixing synthetic proteins of the invention obtained in the methods described above with a pharmaceutically acceptable carrier and further constituents like adjuvant as described above and below.

In a further aspect the invention relates to a composition comprising a synthetic protein obtained in a method described above. The synthetic protein preferably is as defined above. The compositions comprising synthetic proteins as obtained by chemical synthesis in the methods of the invention have several advantages over conventional compositions comprising antigenic proteins obtained from biological systems. Inherently processes for producing proteins in biological systems are subject to biological variation. In addition to this variation, there is the inherent risk that components isolated from biological sources, be it from recombinant hosts, non-GMO or even cell-free systems, may be contaminated with other biomolecules from the biological production system some of which may be very harmful. In contrast, the compositions of the invention are substantially free of biological contaminants. Preferably, the compositions are free biological contaminants to the extent that the level of such contaminants is below the detection level of available assays. The biological contaminants that are absent from the compositions include any biomolecule from the production organism or system. Such contaminants thus include proteins, carbohydrates, nucleic acids including DNA and RNA, lipids and combinations thereof such as e.g. bacterial endotoxins, and may even include viruses that can infect humans. Preferably, the compositions are substantially free of contaminants (biomolecules) from the organism or virus from which the naturally occurring protein is derived, i.e in which the protein occurs naturally, or alternatively, in which the protein is produced in case of recombinant production. A major problem is the potential contamination of the proteinaceous preparation with nucleic acids, in particular pathogenic and/or oncogenic nucleic acids such as e.g. viral and/or recombinant nucleic acids. The compositions comprising the synthetic proteins according to the current invention are thus essentially free of nucleic acid contamination, more in particular free of viral and/or oncogenic nucleic acids, of which in particular DNA. Contamination of nucleic acids and in particular of viral and/or oncogenic nucleic acids, more in particular of HPV nucleic acids can be demonstrated by PCR amplification techniques known in the art (Ausubel et al., Current protocols, 1989). Preferably, the composition comprises a synthetic protein (the protein e.g. comprising an amino acid sequence that is at least 80% identical to at least 46 contiguous amino acids of of a naturally occurring antigenic protein of a pathogen or tumor), whereby the composition is free of a nucleic acid encoding the amino acid sequence of the synthetic protein. The compositions comprising synthetic proteins according to the current invention preferably do not display detectable DNA contamination when assayed by PCR amplification with primers suitable for amplification of viral and/or oncogenic nucleic acids after 30 cycles, more preferably after 40 cycles and most preferably after 50 cycles of PCR amplification. More preferably, the compositions are free of HPV nucleic acid (DNA) fragments as detectable by 30, 40 or 50 cycles of PCR amplification with suitable HPV specific primers. Most preferably, the composition is substantially free of DNA encoding the amino acid sequences of SEQ ID NO.'s 1-6.

A further preferred composition comprises an adjuvant. The adjuvant preferably is an adjuvant that is capable of activating dendritic cells. More preferably the adjuvant is an adjuvant that is capable of activating a TLR, such as defined above. The composition may comprise an adjuvant mixed with the synthetic protein or it may contain an adjuvant that is covalently conjugated to the protein or it may contain both. Moreover more than one adjuvant may be present. A number of adjuvants are well known to one skilled in the art. Suitable adjuvants include incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn -glycero-3-hydroxyphosphoryloxy)-ethylamine (CGP 19835A, referred to as MTP-PE), and RIBI, which contains three components extracted from bacteria, monophosphoryl lipid A, trehalose dinycolate and cell wall skeleton (MPL+TDM+CWS) in a 2% squalene/Tween 80 emulsion. The effectiveness of an adjuvant may be determined by measuring the amount of antibodies directed against the immunogenic peptide. A particularly useful adjuvant and immunisation schedule are described in Kwak et al. New Eng. J. Med. 327-1209-1215 (1992). The immunological adjuvant described there comprises 5% (wt/vol) squalene, 2.5% Pluronic L121 polymer and 0.2% polysorbate in phosphate buffered saline. In a preferred embodiment of the invention an adjuvant is recognized by a Toll-like-receptor (TLR) present on antigen presenting cells, i.e. the adjuvant is a TLR-ligand. Various adjuvants recognized by TLR's are described above and may be used for mixing with and/or conjugation to the synthetic proteins of the invention.

Most preferably the adjuvant is a synthetic adjuvant which is chemically synthesized rather than purified from a biological source. In the most preferred embodiment of the current invention synthetic proteins are conjugated to a (one or more) synthetic TLR recognizable adjuvant(s), which forms the basis for a completely defined and synthetic composition for vaccination. Yet a further preferred composition comprises an anti-CD40 antibody.

The compositions of the invention further comprises a pharmaceutically acceptable carrier. The pharmaceutical compositions (for vaccination) are intended for parenteral, oral, transdermal or transmucosal administration. The pharmaceutical compositions may be administered parenterally, e.g., subcutaneously, intradermally, or intramuscularly. Thus, the invention provides compositions for oral or preferably parenteral administration, which comprise a solution of the immunogenic synthetic proteins or synthetic vaccines dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well-known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as buffering agents, tonicity adjusting agents, wetting agents and the like, for example, sodium acetate, sodium lactate, sodium chloride, potassium chloride, calcium chloride, sorbitan monolaurate, and triethanolamine oleate.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more synthetic proteins of the invention, and more preferably at a concentration of 25%-75%. As noted above, the compositions are intended to induce an immune response to the synthetic proteins. Thus, compositions and methods of administration suitable for maximizing the immune response are preferred. For instance, synthetic proteins may be introduced into a host, including humans, linked to a carrier or as a homopolymer or heteropolymer of active protein units. Useful carriers are well known in the art, and include, e.g., thyroglobulin, albumins such as human serum albumin, tetanus toxoid, polyamino acids such as poly(lysine:glutamic acid), influenza, hepatitis B virus core protein, hepatitis B virus recombinant vaccine and the like. Alternatively, a “cocktail” of synthetic proteins can be used, comprising a selection of immunogenic HPV synthetic proteins, such as E7, E6 and E2 or parts thereof and their conjugates with adjuvants. A mixture of more than one synthetic protein has the advantage of increased immunological reactions.

The concentration of immunogenic synthetic proteins of the invention in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990). E.g. transdermal delivery systems include patches, gels, tapes and creams, and can contain excipients such as solubilizers, permeation enhancers (e.g. fatty acids, fatty acid esters, fatty alcohols and amino acids), hydrophilic polymers (e.g. polycarbophil and polyvinyl pyrillidine and adhesives and tackifiers (e.g. polyisobutylenes, silicone-based adhesives, acrylates and polybutene). Transmucosal delivery systems include patches, tablets, suppositories, pessaries, gels, and creams, and can contain excipients such as solubilizers and enhancers (e.g. propylene glycol, bile salts and amino acids), and other vehicles (e.g. polyethylene glycol, fatty acid esters and derivatives, and hydrophilic polymers such as hydroxypropylmethyl cellulose and hyaluronic acid). Injectable delivery systems include solutions, suspensions, gels, microspheres and polymeric injectables, and can comprise excipients such as solubility-altering agents (e.g. ethanol, propylene glycol and sucrose) and polymers (e.g. polycaprylactones, and PLGA's). Implantable systems include rods and discs, and can contain excipients such as PLGA and polycapryl lactone. Other delivery systems that can be used for administering the pharmaceutical composition of the invention include intranasal delivery systems such as sprays and powders, sublingual delivery systems and systems for delivery by inhalation. For administration by inhalation, the pharmaceutical compositions of the present invention are conveniently delivered in the form of an aerosol spray presentation from pressurised packs or a nebuliser, with the use of a suitable propellant, e.g., dichlorodifluoromethane, trichlorofluoromethane, dichlorotetrafluoroethane, carbon dioxide or other suitable gas. In the case of a pressurised aerosol the dosage unit may be determined by providing a valve to deliver a metered amount. Capsules and cartridges of, e.g., gelatin for use in an inhaler or insufflator may be formulated containing a powder mix of the peptides of the invention and a suitable powder base such as lactose or starch. The pharmaceutical compositions of the invention may be further formulated for administration by inhalation as e.g. described in U.S. Pat. No. 6,358,530. A preferred composition of the invention is a composition for use as a vaccine.

In yet another aspect the invention relates to a method for the treatment or prevention of a tumor or an infectious disease, whereby the method comprises the administration to a subject of a synthetic protein produced as defined above or a composition as defined above, in a therapeutically effective amount. Thereby the synthetic protein comprises an amino acid sequence of a naturally occurring antigenic protein of the tumor or infectious agent as defined above. A preferred method is a method for the treatment or prevention of an HPV-associated disease, including prevention or treatment of HPV infection, particularly genital infection, prevention or regression of HPV virus-induced lesions such as genital warts and epithelial dysplasia, and particularly including the prevention and treatment HPV-induced dysplasia and transformation into cancer, in particular cervival cancer, anogenital cancer or head- and neck-cancer, and other HPV-induced cancers.

For those jurisdictions where methods of treatment are unpatentable by law, the invention likewise pertains to the use of a synthetic protein produced as defined above, or the use of a composition as defined above, for the manufacture of a medicament for (use in a method for) the treatment or prevention of a tumor or an infectious disease as defined above.

The invention thus relates to the use of synthetic proteins and synthetic compositions for vaccination in a method for vaccination of a subject. Preferably the method of vaccination is directed against HPV, more preferably against the oncogenic types HPV 16, 18, 31, 33 and 45. The method comprises administering to the subject a pharmaceutical composition comprising a synthetic protein as defined above. In a preferred method, the pharmaceutical composition also comprises a TLR activating adjuvant, preferably a synthetic adjuvant and-more preferably conjugation of the synthetic adjuvant to the synthetic protein of the invention.

In another aspect, the invention relates to the use of a HPV-derived antigenic synthetic protein as defined above for the manufacture of a vaccine or composition for vaccination, aimed at prophylaxis and/or therapy of HPV infection in a subject Preferably, the vaccine is a pharmaceutical composition suitable for oral, parenteral, transdermal or transmucosal administration.

Pharmaceutical (vaccine) compositions comprising at least one synthetic protein of the invention are also an embodiment of the invention. The synthetic proteins of the present invention and pharmaceutical compositions thereof are useful for administration to mammals, particularly humans, to treat and/or prevent disease as indicated above. Suitable formulations are found for instance in Remington's Pharmaceutical Sciences, Mack Publishing Company, Philadelphia, Pa., 17th ed. (1985), which is well known in the art and may easily be adapted by a skilled artisan.

The immunogenic synthetic proteins of the invention are administered prophylactically or to an individual already suffering from the disease. The compositions are administered to a patient in an amount sufficient to elicit an effective immune response. An amount adequate to accomplish this is defined as “therapeutically effective dose” or “immunogenically effective dose.” Amounts effective for this use will depend on, e.g., the peptide composition, the manner of administration, the stage and severity of the disease being treated, the weight and general state of health of the patient, and the judgement of the prescribing physician, but generally range for the initial immunisation (that is for therapeutic or prophylactic administration) from about 0.1 μg to about 50 μg per kilogram (kg) of body weight per patient, more commonly from about 1 μg to about 20 μg per kg of body weight. Boosting dosages are typically from about 1 μg to about 20 μg per kg of body weight, using a boosting regimen over weeks to months depending upon the patient's response and condition. A suitable protocol would include 3-4 priming injections at three weeks intervals, eventually followed by booster injections at regular intervals (e.g. 6 months).

The following non-limiting Examples describe the preparation of HPV-derived antigenic synthetic proteins of the invention.

### DESCRIPTION OF THE FIGURES

FIG. 1: Synthetic scheme for the synthesis of the HPV16-E7 protein.

FIG. 2: Maldi-tof mass spectrum of purified synthetic HPV16-E7 protein.

FIG. 3: A single injection of 4.5 nmol synthetic HPV16-E7 protein results in the induction of high numbers of HPV16-E7₄₉₋₅₇-specific CD8+ T-cells. C57BL/6 mice were injected sub-cutaneously with the indicated antigens at equimolar concentrations of the minimal CTL epitope (E749-57) mixed with CpG, with 150 μg of E7₄₃₋₇₇ (high dose) peptide+CpG which served as positive control, or not vaccinated (naive). The percentage of spleen-derived CD8+ T-cells stained with H-2D^{b}-E7₄₉₋₅₇ PE-conjugated tetramers (TM) is shown directly ex-vivo (A) or following one round of in vitro stimulation (B). Bars indicate the mean percentage of three mice and SEM.

FIG. 4: Vaccination with synthetic HPV16-E7 protein induces functional IFNγ-producing CD8+ T-cells. Spleen cell cultures were stimulated with the dendritic cell line D1 only or D1 cells pulsed with the E7₄₃-₇₇ peptide or D1 pulsed with recombinant HPV16-E7 protein and analyzed by intracellular cytoline staining on a flow cytometer. Three mice injected with the synthetic HPV16-E7 protein are shown in combination with a representative example of mice injected with an equimolar concentration of recombinant HPV16-E7 or a high dose of peptide E7₄₃₋₇₇ and a non-vaccinated (nave) mouse.

FIG. 5

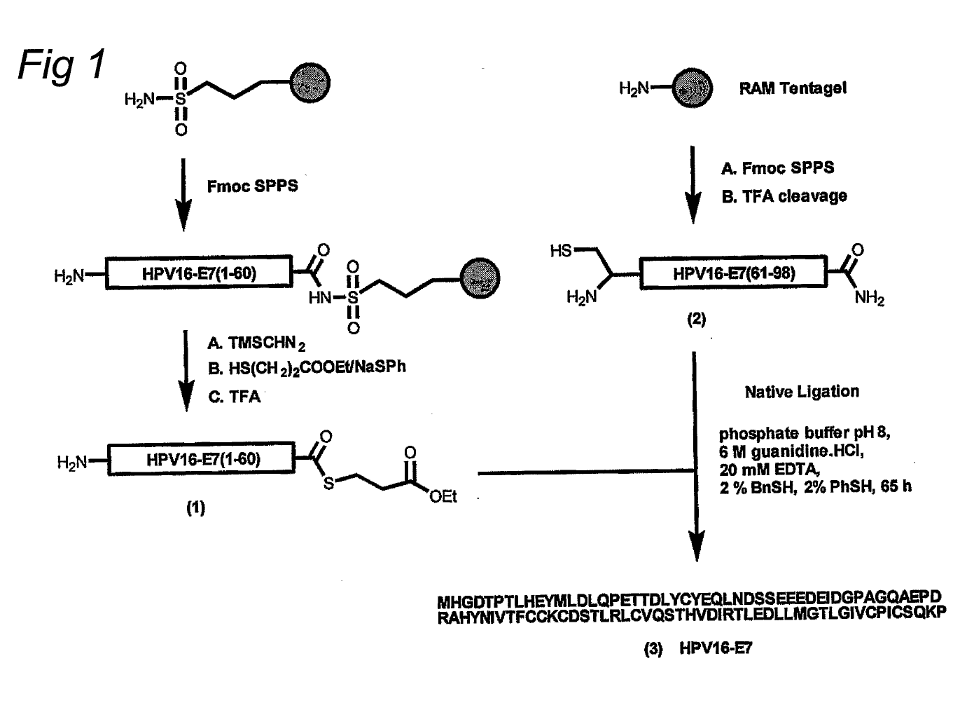

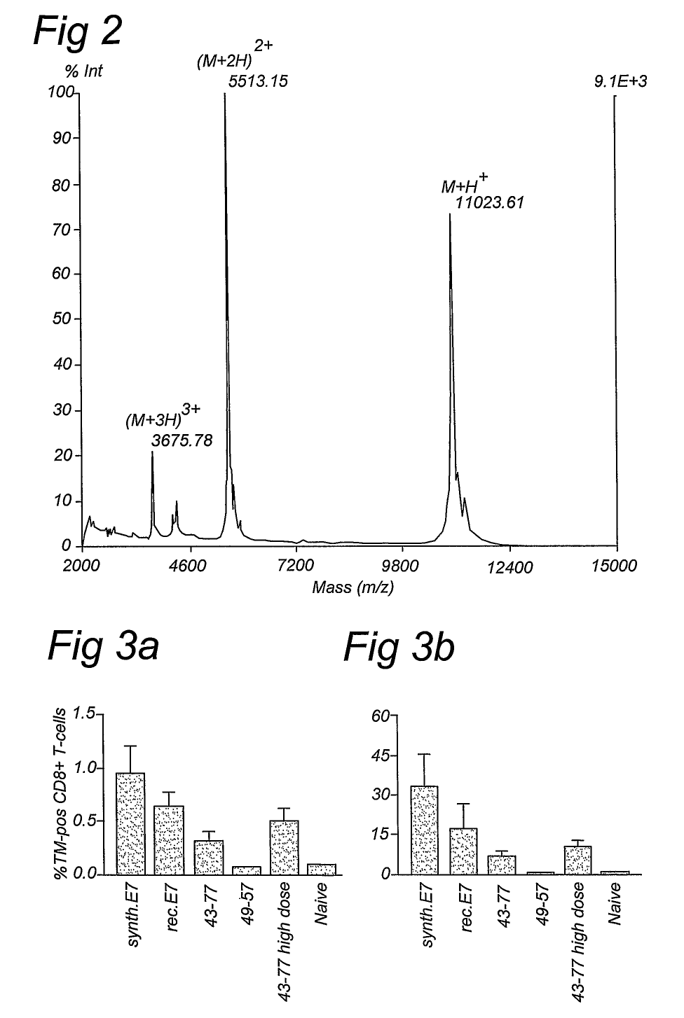

a) Groups of mice were vaccinated at day −28 and day −14 with 4.5 nmol of synthetic E7 protein (n=10) and equimolar amounts of recombinant E7 protein (n=9) or long peptide admixed with 50 μg CpG (peptide low dose, n=10) or with PBS only (naive, n=7). As a control the long peptide was also used at an approximately 8-fold higher dose (n=5) which was previously established to be protective in 80% of mice. At day 0, 50.000 TC-1 tumour cells were s.c. injected at the contralateral side and tumour growth was monitored every 2-3 days up to 100 days and survival curves were plotted. b) Mice challenged with TC-1 tumour cells were vaccinated at the contralateral side with indicated vaccines (n=8 per group) when tumours were palpable at day 9 and boosted at day 23 (arrows). Tumour growth was monitored until day 95 and survival curves were plotted.

### EXAMPLES

### Example 1

### Chemical Synthesis of HPV16 E7 Peptides and Ligation of Peptides Chemical Synthesis of HPV 16 E7 Peptides and Protein

The homogeneous synthetic E7 protein was prepared by chemical ligation of two oligopeptides assembled separately by Fmoc solid phase synthesis. The N-terminal 60-meric segment of E7 was prepared on sulphonamide safety-catch resin and converted into thioester 1 [E7 (1-60)] according to a published procedure (Ingenito, R et al., J. Am. Chem. Soc. 121, 11369-11374 (1999). The C-terminal 38-meric carboxamide [E7(61-98), 2] was produced via a standard Fmoc solid phase protocol. Subsequently, the RP-HPLC purified fragments 1 and 2 were ligated to give the full-length E7 protein (3). The ligation reaction could be successfully conducted using thiophenol/benzyl mercaptane as additives according to the one of the original native ligation procedures (Hackeng T. M., et al, Proc. Natl. Acad. Sci. USA 96, 10068-10073 (1999) and Dawson, P. E., et al., J. Am. Chem. Soc. 119, 4325-4329 (1997). We found, however, mercaptoethyl sulphonic acid sodium salt (Flavell R. R., et al., Org. Lett. 4, p165-168 2002) to be a more convenient additive as it gave a homogeneous and odourless reaction mixture.

The following essential modifications were introduced to the overall synthetic pathway in order to obtain an acceptable yield of the protein. First, it proved to be necessary to include 20 mM EDTA into the ligation buffer to prevent complete and irreversible precipitation of the product/reaction intermediates. We have also found that the preparative purification of the E7 protein could not been done with RP HPLC because of the poor yield over this step. A satisfactory purification protocol comprises gel filtration of the reaction mixture over Superdex 75 column under strongly denaturing conditions (6 M guanidine hydrochloride) followed by dialysis of the pooled fractions containing the target protein against water. It has to be noted that gel filtration step performed under non-denaturing (phosphate buffer) or weakly denaturing (7 M urea) condition yielded the product still contaminated with E7(61-98) presumably because of co-aggregation of the latter fragment and full-length E7. These results are fully consistent with the well known (Alonso, L. G., et al., Biochemistry, 41, 10510-10518 (2002) aggregation potential of E7 and the reported observation that it is the C-terminal part of E7 which is responsible for the oligomerization of the protein (Clemens, K. E., et al., Virol. 214, 289-293 (1995).

### Experimental

Peptide synthesis—Fragments E7(1-60) thioester (1) and E7(61-98) C-terminal carboxamide (2) were prepared by Fmoc-SPPS with PyBOP activation on a 0.25 mmol scale using ABI 433A instrument. An in situ protocol suggested in (Han, Y. X., et al., J. Org. Chem. 62, 4307-4312 (1997) in order to minimize racemisation of the Cys residues was used. Each synthetic cycle comprised a Fmoc removal with 20% piperidine in NW, a NMP wash, a 1 h PyBOP assisted coupling step with 1 mmol of a Fmoc-amino acid in the presence of 2 mmol of DIPEA, second NMP wash, Ac₂O/DIPEA capping step and, finally, a NMP wash.

Peptide thioester E7(1-60) (1)—Fragment 1 was assembled starting from 4-sulfamylbutyryl AM resin (Novabiochem). The N-terminal methionine residue was introduced as Boc-Met-OH. The thioester was generated from the immobilised and protected oligopeptide. Briefly, the resin was alkylated with TMSCHN₂ and the peptide was cleaved with ethyl 3-mercaptopropionate in the presence of NaSPh and deprotected with TFA/ethyl 3-mercaptopropionate/TIS/m-cresol/H₂O 96/1.2/1.2/0.8/0.8.

Peptide amide E7(61-98) (2)—Fragment 2 was assembled on RAM Tentagel resin (Rapp Polymere). Upon the completion of the chain assembly the peptide was deprotected and cleaved from the resin by 2 h treatment with a cleavage mixture (TFA/EDT/TIS/m-cresol/H₂O 96/1.2/1.2/0.8/0.8) as described (Dick, F. in Methods in Molecular Biology, Vol. 35: Peptide Synthesis Protocols pp. 63-72. (Eds. Pennington, M. W.& Dunn, B. M.) (Humana Press Inc., Totowa, N.J., 1994)).

Ligation to obtain full-length E7-protein (3)

Method A Peptide thioester 1 (22 mg, 3.1 μmol) was mixed with peptide 2 (11.3 mg, 2.7 μmol) and dissolved in 3.0 mL buffer (100 mM phosphate, 20 mM EDTA, 6 M Gdn·HCl, pH 8). Subsequently, 60 μL thiophenol and 60 μL benzyl mercaptane were added and the mixture was stirred for 65 h at 22° C. The mixture was treated with 100 mg DTT and loaded on Superdex 75 HL 16/60 column (Pharmacia) equilibrated in 25 mM Na₂HPO₄, 25 MM NaH₂PO₄, 5 mM EDTA, 5 mM DTT, pH 7. The product eluting at 37 mL (1 mL/min) was collected and repurified by RP HPLC (Vydac C-4 214TP510 column; 250×10 mm, 5 mL/min) eluting with a gradient of acetonitrile in 0.1% aq. TFA) to give 0.6 mg (yield 2%) of homogeneous E7-protein as determined by mass-spectroscopy, RP HPLC and quantitative amino acid analysis. Electrospray ionisation MS was performed on PE Sciex API 165 single quadrupole mass spectrometer. Matrix-assisted laser-desorption ionization (VERDI) MS-spectra were recorded on a Voyager-DE PRO instrument using a-cyano-4-hydroxycinnamic acid as a matrix.

Method B Peptide thioester 1 (10.5 mg, 1.4 μmol) was mixed with peptide 2 (16 mg, 2 μmol) and dissolved in 1.8 mL of the ligation buffer (200 mM phosphate, 20 mM EDTA, 6 M guanidine hydrochloride, 75 mM MesNa, pH 8.5) and the mixture was stirred for 65 h at 22° C. and then loaded on Superdex 75 HL 16/60 column (Pharmacia) equilibrated in the purification buffer (6 M guanidine hydrochloride, 25 mM Na₂HPO₄, 25 mM NaH₂PO₄, 5 mM EDTA, 5 mM DTT, pH 7) and eluted at 0.8 mL/min. The product eluting at 50 mL was collected (5.5 mL) and dialyzed into water using dialysis tubing with 3 kD cut-off (2 L water, changed 3 times over 40 h) to give 7.5 mL of a solution containing 6.7 mg (yield 44%) of E7 protein. The chromatographic, spectroscopic and immunogenic properties of this material were indistinguishable from the protein obtained by method A.

### Example 2

### Chemical Synthesis of HPV16 E6

| HPV16-E6 PROTEIN SEQUENCE | |
|---|---|
| 001 | MHQKRTAMFQ DPQERPRKLP QLCTELQTTI HDIILECVYC KQQLLRREVY DFAFRDLCIV |
| 061 | YRDGNPYAVC DKCLKFYSKI SEYRHYCYSL YGTTLEQQYN KPLCDLLIRC INCQKPLCPE |
| 121 | EKQRHLDKKQ RFHNIRGRWT GRCMSCCRSS RTRRETQL |

Four fragments selected for peptide synthesis to obtain full length HPV16E6 synthetic protein:

| | |
|---|---|
| 01:001-039 | MHQKRTAMFQDPQERPRKLPQLCTELQTTIHDIILE |
| | CVY-SR |
| 02:040-072 | X-CKQQLLRREVYDFAFRDLCIVYRDGNPYAVCDK- |
| | SR |
| 03:073-117 | X-CLKFYSKISEYRHYCYSLYGTTLEQQYNKPLCDLL |
| | IRCINCQKPL-SR |
| 04:118-158 | CPEEKQRHLDKKQRFHNIRGRWTGRCMSCCRSSRTRR |
| | ETQL-OH |

The fragments are depicted as their thioesters (-SR) wherein X represents a temporary protecting group that is removed after the particular fragment has been coupled (e.g. the MSC-group). The synthesis process is: coupling fragment 04 to fragment 03, followed by removal X from construct 03/04, coupling fragment 03/04 to fragment 02, removal X from construct 02/03/04, coupling fragment 02/03/04 to fragment 01.

### Example 3

### Chemical Synthesis of HPV16 E2

| HPV16-E2 PROTEIN SEQUENCE | |
|---|---|
| 001 | METLCQRLNV CQDKILTHYE NDSTDLRDHI DYWKHMRLEC AIYYKAREMG FKHINHQVVP |
| 061 | TLAVSKNKAL QAIELQLTLE TIYNSQYSNE KWTLQDVSLE VYLTAPTGCI KKHGYTVEVQ |
| 121 | FDGDICNTMH YTNWTHIYIC EEASVTVVEG QVDYYGLYYV HEGIRTYFVQ FKDDAEKYSK |
| 181 | NKVWEVHAGG QVILCPTSVF SSNEVSSPEI IRQHLANHPA ATHTKAVALG TEETQTTIQR |
| 241 | PRSEPDTGNP CHTTKLLHRD SVDSAPILTA FNSSHKGRIN CNSNTTPIVH LKGDANTLKC |
| 301 | LRYRFKKHCT LYTAVSSTWH WTGHNVKHKS AIVTLTYDSE WQRDQFLSQV KIPKTITVST |
| 361 | GFMSI |

Seven fragments selected for peptide synthesis to obtain full length HPV16 E2 synthetic protein:

| | |
|---|---|
| 01:001-039 | METLCQRLNVCQDKILTHYENDSTDLRDHIDYWKHMRL |
| | E-SR |
| 02:040-108 | X-CAIYYKAREMGFKHINHQVVPTLAVSKNKALQAIEL |
| | QLTLETIYNSQYSNEKWTLQDVSLEVYLTAPTG-SR |
| 03:109-139 | X-CIKKHGYTVEVQFDGDICNTMHYTNWTHIYI-SR |
| 04:140-194 | X-CEEASVTVVEGQVDYYGLYYVHEGIRTYFVQFKDDA |
| | EKYSKNKVWEVHAGGQVIL-SR |
| 05:195-250 | X-CPTSVFSSNEVSSPEIIRQHLANHPAATHTKAVALG |
| | TEETQTTIQRPRSEPDTGNP-SR |
| 06:251-299 | X-CHTTKLLHRDSVDSAPILTAFNSSHKGRINCNSNTT |
| | PIVHLKGDANTLK-SR |
| 07:300-365 | CLRYRFKKHCTLYTAVSSTWHWTGHNVKHKSAIVTLTY |
| | DSEWQRDQFLSQVKIPKTITVSTGFMSI |

The fragments are depicted as their thioesters (-SR), wherein X represents a temporary protecting group that is removed after the particular fragment has been coupled (e.g. the MSC-group). The synthesis strategy is comparable with HPV16 E6.

Note: coupling between construct 06/07 and fragment 05 can be difficult because this coupling is to a P.

HPV16-E2, alternative strategy, two overlapping parts of the protein, glycine-ligation.

Normally chemical ligation proceeds by coupling of an C-terminal thioester of a fragment to an N-terminal C of another fragment, due to lenght restrictions and/or an unfavourable sequence. In case of HPV16E2 an alternative strategy can be used:

PART 1: 001-210

| | |
|---|---|
| 01:001-039 | METLCQRLNV CQDKILTHYE NDSTDLRDHI DYWKH |
| | MRLE-SR |
| 02:040-108 | X-CAIYYKAREMGFKHINGQVVPTLAVSKNKALQAIEL |
| | QLTLETIYNSQYSNEKWTLQDVSLEYLTAPTG-SR |
| 03:109-155 | X-CIKKHGYTVEVQFDGDICNTMHYTNWTHIYICEEAS |
| | VTVVEGQVDYY-SR |
| 04:156-210 | XX-GLYYVHEGIRTYFVQFKDDAEKYSKNKVWEVHAGG |
| | QVILCPTSVFSSNEVSSPEI |

PART 2: 190365

| | |
|---|---|
| 01:190-229 | GQVILCPTSVFSSNEVSSPEIIRQHLANHPAATHTKAV |
| | AL-SR |
| 02:230-280 | XXGTEETQTTIQRPRSEPDTGNPCHTTKLLHRDSVDSA |
| | PILTAFNSSHKGRIN-SR |
| 03:281-308 | X-CNSNTTPIVHLKGDANTLKCLRYRFKKH-SR |
| 04:309-365 | CTLYTAVSSTWHWTGHNVKHKSAIVTLTYDSEWQRDQF |
| | LSQVKIPKTITVSTGFMSI |

Fragments are depicted as their thioesters (-SR) wherein X represents a temporary protecting group that is removed after the particular fragment has been coupled (e.g. the MSC-group), XX-represents an S-protected N-(2,3,4-trimethoxy-5-mercaptophenyl)group attached to the N-terminal G [J. Am. Chem. Soc. 124, 4642 (2002)].

### Example 4

### Chemical Synthesis of HPV18 E7

| HPV18-E7 PROTEIN SEQUENCE | |
|---|---|
| 01 | MHGPKATLQD IVLHLEPQNE IPVDLLCHEQ LSDSEEENDE IDGVNHQHLP ARRAEPQRHT |
| 61 | MLCMCCKCEA RIELVVESSA DDLRAFQQLF LNTLSFVCPW CASQQ |

Two fragments selected for peptide synthesis to obtain full length HPV18 E2 synthetic protein, details identical to example 1:

| | |
|---|---|
| 01:001-065 | MHGPKATLQDIVLHLEPQNEIPVDLLCHEQLSDSEEEN |
| | DEIDGVNHQHLPARRAEPQRHT MLCMC-SR |
| 02:066-099 | CKCEA RIELVVESSA DDLRAFQQLF LNTLSFVCPW |
| | CASQQ |

### Example 5

### Chemical Synthesis of HPV18 E6

| HPV18-E6 PROTEIN SEQUENCE | |
|---|---|
| 001 | MARFEDPTRR PYKLPDLCTE LNTSLQDIEI TCVYCKTVLE LTEVFEFAFK DLFVVYRDSI |
| 061 | PHAACHKCID FYSRIRELRH YSDSVYGDTL EKLTNTGLYN LLIRCLRCQK PLNPAEKLRH |
| 121 | LNEKRRFHNI AGHYRGQCHS CCNRARQERL QRRRETQV |

Four fragments selected for peptide synthesis to obtain full length HPV18 E6 synthetic protein:

| | |
|---|---|
| 01:001-034 | MARFEDPTRRPYKLPDLCTELNTSLQDIEITCVY-SR |
| 02:035-064 | X-CKTVLELTEVFEFAFKDLFVVYRDSIPHAA-SR |
| 03:065-104 | X-CHKCIDFYSRIRELRHYSDSVYGDTLEKLTNTGLYN |
| | LLIR-SR |
| 04:105-158 | CLRCQKPLNPAEKLRHLNEKRRFHNIAGHYRGQCHSCC |
| | NRARQERLQRRRETQV |

Fragments are depicted as their thioesters (-SR), wherein X represents a temporary protecting group that is removed after the particular fragment has been coupled (e.g. the MSC-group).

### Example 6

### Chemical Synthesis of HPV18 E2

| HPV18-E2 PROTEIN SEQUENCE | |
|---|---|
| 001 | MQTPKETLSE RLSCVQDKII DHYENDSKDI DSQIQYWQLI RWENAIFFAA REHGIQTLNH |
| 061 | QVVPAYNISK SKAHKAIELQ MALQGLAQSR YKTEDWTLQD TCEELWNTEP THCFKKGGQT |
| 121 | VQVYFDGNKD NCMTYVAWDS VYYMTDAGTW DKTATCVSHR GLYYVKEGYN TFYIEFKSEC |
| 181 | EKYGNTGTWE VHFGNNVIDC NDSMCSTSDD TVSATQLVKQ LQHTPSPYSS TVSVGTAKTY |
| 241 | GQTSAATRPG HCGLAEKQHC GPVNPLLGAA TPTGNNKRRK LCSGNTTPII HLKGDRNSLK |
| 301 | CLRYRLRKHS DHYRDISSTW HWTGAGNEKT GILTVTYHSE TQRTKFLNTV AIPDSVQILV |
| 361 | GYMTM |

Seven fragments selected for peptide synthesis to obtain fill length HPV18 E2 synthetic protein:

| | |
|---|---|
| 01:001-013 | MQTPKETLSERLS-SR |
| 02:014-101 | X-CVQDKIIDHYENDSKDIDSQIQYWQLIRWENAIFFA |
| | AREHGIQTLNHQVVPAYNISKSKAHKAIELQMALQGLA |
| | QSRYKTEDWTLQDT-SR |
| 03:102-155 | X-CEELWNTEPTHCFKKGGQTVQVYFDGNKDNCMTYVA |
| | WDSVYYMTDAGTWDKTAT-SR |
| 04:156-199 | X-CVSHRGLYYVKEGYNTFYIEFKSECEKYGNTGTWEV |
| | HFGNNVID-SR |
| 05:200-251 | X-CNDSMCSTSDDTVSATQLVKQLQHTPSPYSSTVSVG |
| | TAKTYGQTSAATRPGH-SR |
| 06:252-300 | X-CGLAEKQHCGPVNPLLGAATPTGNNKRRKLCSGNTT |
| | PIIHLKGDRNSLK-SR |
| 07:301-365 | X-CLRYRLRKHSDHYRDISSTWHWTGAGNEKTGILTVT |
| | YHSETQRTKFLNTVAIPDSVQILVGYMTM |

Fragments are depicted as their thioesters (-SR) wherein X represents a temporary protecting group that is removed after the particular fragment has been coupled (e.g. the MSC-group).

HPV18-E2, alternative strategy, two overlapping parts of the protein, glycine-ligation.

Preferably chemical ligation proceeds by coupling of an C-terminal thioester of a fragment to an N-terminal C of another fragment, due to lenght restrictions and/or an unfavourable sequence. In case of HPV16E2 an alternative strategy can be used:

PART 1: 001-210

| | |
|---|---|
| 01:001-053 | MQTPKETLSERLSCVQDKIIDHYENDSKDIDSQIQYWQ |
| | LIRWENAIFFAAREH-SR |
| 02:054-112 | XX-GIQTLNHQVVPAYNISKSKAHKAIELQMALQGLAQ |
| | SRYKTEDWTLQDTCEELWNTEPTH-SR |
| 03:113-155 | X-CFKKGG VQVYFDGNKD NCMTYVAWDS |
| | VYYMTDAGTW DKTAT-SR |
| 04:156-210 | X-CVSHRGLYYVKEGYN TFYIEFKSEC |
| | EKYGNTGTWE VHFGNNVIDC NDSMCSTSDD |

PART 2: 191-365

| | |
|---|---|
| 01:191-251 | VHFGNNVIDCNDSMCSTSDDTVSATQLVKQLQHTPSPY |
| | SSTVSVGTAKTYGQTSAATRPGH-SR |
| 02:252-300 | X-CGLAEKQHCGPVNPLLGAATPTGNNKRRKLCSGNTT |
| | PIIHLKGDRNSLK-SR |
| 03:301-365 | X-CLRYRLRKHSDHYRDISSTWHWTGAGNEKTGILTVT |
| | YHSETQRTKFLNTVAIPDSVQILVGYMTM |

Fragments are depicted as their thioesters (-SR) wherein X represents a temporary protecting group that is removed after the particular fragment has been coupled (e.g. the MSC-group) and XX-represents an S-protected N-(2,3,4-trimethoxy-5-mercaptophenyl)group attached to the N-terminal G [J. Am. Chem. Soc. 124, 4642 (2002)].

### Example 7

### Antigenicity of Synthetic HPV16-E7 Protein Vaccine

### Methods

Control antigens and adjuvants. Two peptides were generated, the H-2D^{b}-restricted CTL epitope HPV16-E7₄₉₋₅₇ (RTF) and the E7₄₃₋₇₇35 residue long peptide GQAEPDRAHYNIVTFCCKCDSTLRLCVQSTHVDIR. The purity of the peptides was determined by RP-HPLC and was found to be routinely over 90% pure. Peptides were dissolved in 0.5% DMSO in PBS and, if not used immediately, stored at −20° C. The recombinant was produced in recombinant E. coli transformed with Pet-19b-HPV16-E7 and purified as described previously (De Bruijn, M. L. et al., Cancer Res. 58 p 724-31, 1999). CpG-oligodeoxynucleotides (ODN) 1826, sequence TTCATGACGTTCCTGACGTT, were provided by Coley Pharmaceutical and used at a working concentration of 50 μg/mouse (Zwaveling S. et al., J. Immunol. 169, p350-8, 2002).

Mice, immunizations, cell lines and T-cell cultures. C57BL/6 (B6, H-2^{b}) mice were obtained from IFFA Credo (Paris, France). Tumor cell line 13.2 was derived from MEC (B6) transformed with adenovirus type 5-derived E1 protein in which the H-2D^{b} E1A epitope was replaced with the HPV16-E7₄₉₋₅₇ CTL epitope. TC-1, which was derived from primary epithelial cells of C57BL/6 mice cotransformed with HPV-16 E6 and E7 and c-Ha-ras oncogenes were cultured in IMDM+10% FCS (Van der Burg S. H. et al., Vaccine 19, 3652-60, 2001). Dl cells are long-term growth factor dependent immature splenic dendritic cells (DC) derived from C57BL/6 mice and were cultured as described (Winzler C. et al., J. Exp. Med. 185, p317-28, 1997). C57BL/6 mice were injected subcutaneously with equimolar levels (4.5 nmol) of either the E7₄₉₋₅₇ short peptide (5.0 μg), or E7₄₃₋₇₇ 35-residue long peptide (18 μg), recombinant HPV16-E7 (50 μg) or synthetic HPV16-E7 protein (50 μg) dissolved in PBS. ODN-CpG 1826 (50 μg) was dissolved in PBS and mixed with the peptides before subcutaneous vaccination. As a positive control, mice were also injected with an approximately 8-fold higher dose of peptide (37.7 nmol, 150 μg) dissolved in PBS and mixed with ODN-CpG 1826 (Zwaveling et al., J. Immunol. 169, p 350-8, 2002). The total injected volume was 200 μl/mouse. Spleens were harvested after 10 days. T cells were obtained from immunized mice by culturing spleen cells (4×10⁶ cells/well of a 24-wells plate) in complete medium in the presence of 5×10⁵ E7₄₉₋₅₇-expressing cells (tumor cell line 13.2). Cultures were maintained at 37° C. in humidified air containing 5% CO₂. No exogenous IL-2 was added. On day six, dead cells were removed from the culture by centrifugation over a Ficoll density gradient and remaining viable cells were seeded in 24-wells plates at 1.5×10⁶ cells/well. On day seven, tetramer staining or intracellular cytokine staining was performed.

Analysis of HPV16-E7-specific CD8+ T-cells. PE-labeled H-2D^{b} epitope E7₄₉₋₅₇ (RAHYNIVTF)-containing tetramers were constructed and used for the analysis of peptide-specific CTL-immunity as described earlier (Van der Burg et al., Vaccine 19, p 3652-60, 2001). FITC-labeled anti-CD8b.2 Ab (Ly-3.2) (clone 53-5.8) and PE-labeled anti-IFNγ Ab (clone XMG1.2) (BD PharMingen, San Diego, USA) were used for the analysis of antigen-specific IFNγ production of HPV16-E7-specific CTL as described previously (Zwaveling S. et al., J. Immunol. 169 p 350-8, 2002).

Tumour challenge experiments. In prophylactic vaccination strategy experiments the mice were subcutaneously vaccinated at day −28 and day −14 with indicated vaccines at their left flank. At day 0, mice were subcutaneously injected with 50.000 TC-1 tumour cells and tumour growth was followed for 100 days. In the therapeutic vaccination setting mice were challenged at the left flank with 50.000 TC-1 tumourcells. At day 9, when tumours were palpable, mice were vaccinated at the right flank with indicated vaccines. Fourteen days later these mice received a booster injection with the vaccinevaccination. Tumour growth was monitored for 95 days.

In vivo Antigenicity of Synthetic HPV16-E7 Protein

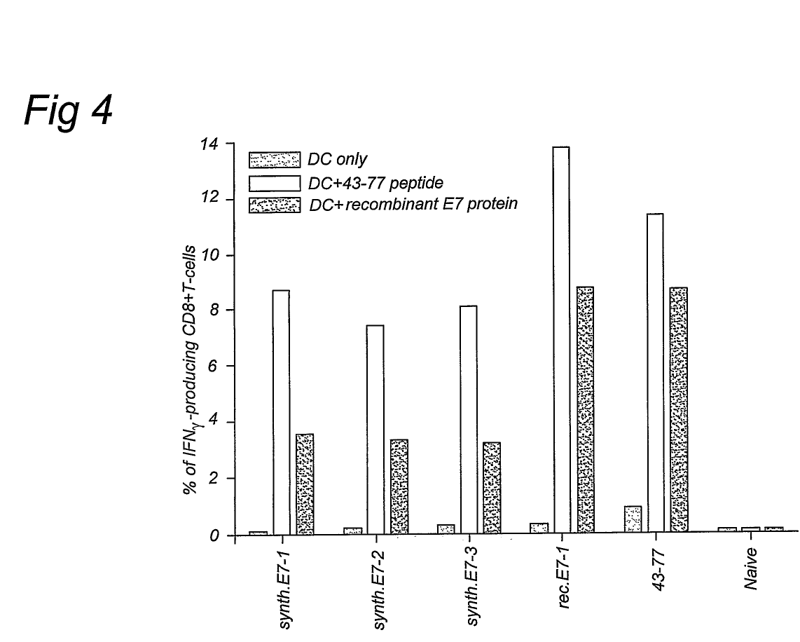

Since numerous studies show that: (1) protection of C57BL/6 mice against HPV16-E7-expressing tumors is largely dependent on E7₄₉₋₅₇-specific CD8+ T cells (De Bruijn M. L. et al., Cancer Res. 58, p 724-31, 1998, Greenstone H. L. et al., PNAS 95, p 1800-5, 1998, Lin K. Y. et al., Cancer Res. 56, p21-6, 1996, Feltkamp M. C. et al., Eur. J. Immunol. 23, p 2242-9,, 1993), and (2) that the ability of HPV16-E7-specific T-cells to protect against tumor development or to eradicate established tumors is correlated with the percentage of E7₄₉₋₅₇-tetramer positive CD8+ T-cells (Van der Burg et al., Vaccine 19, p 3652-60, 2001), the antigenicity of synthetic HPV16-E7 protein was assessed by its capacity to induce such HPV16-E7₄₉₋₅₇-specific CD8+ T-cells. C57BL/6 mice were injected with several vaccines that have been used successfully in the past, including the minimal CTL epitope (E749-57: RAHYNIVTF), a longer peptide CE743-77) that was known to induce vigorous E7₄₉₋₅₇-specific CD8+ T-cell responses, recombinant HPV16-E7 or the synthetic HPV16-E7 protein at equimolar concentrations of the minimal CTL epitope, in combination with CpG. Ten days following vaccination, the spleens were harvested and the cells directly analysed by H2-D^{b} E7₄₉₋₅₇ (RAHYNIVTF)-tetramer staining (Van der Burg S. H. Vaccine 19, p 3652-60, 2001) (FIG. 3a) as well as subjected to an extra round of in vitro stimulation, which magnifies but does not alter the hierarchy of in vivo induced CD8+ T cell responses, before the percentage of E7₄₉₋₅₇ peptide-specific CD8+ T-cells was determined (FIG. 3b). As expected, the longer E7 peptide was able to induce strong HPV16-E7-specific CD8+ T-cells at a high antigen dose as well as at the lower dose, whereas the response induced by the minimal CTL epitope was significantly lower. Importantly, the HPV16-E7-specific CD8+ T-cell response induced by one single injection of synthetic E7 protein was comparable to that of the recombinant HPV16-E7 protein and somewhat higher than the other vaccines. To confirm that functional CD8+ T-cell responses were triggered following a single vaccination with the synthetic E7 protein, the numbers of INFγ-producing CD8⁺ cells were measured upon stimulation with dendritic cells (DC) only, or pulsed with either the long E7₄₃₋₇₇ peptide or the recombinant E7 protein. High numbers of INFγ-producing CD8+ T-cells were detected in the spleens of mice vaccinated with the synthetic E7 protein, confirming that the CD8⁺ T-cells detected by the H2-D^{b} E7₄₉₋₅₇-tetramers were functionally active (FIG. 4). Furthermore, the CD8+ T-cells from these mice reacted against recombinant E7 protein-pulsed DC, indicating that the synthetic HPV16-E7 protein retained its full antigenic potential.

### The Synthetic E7-Protein Vaccination Results in Eradication of TC-1 Tumour Cells

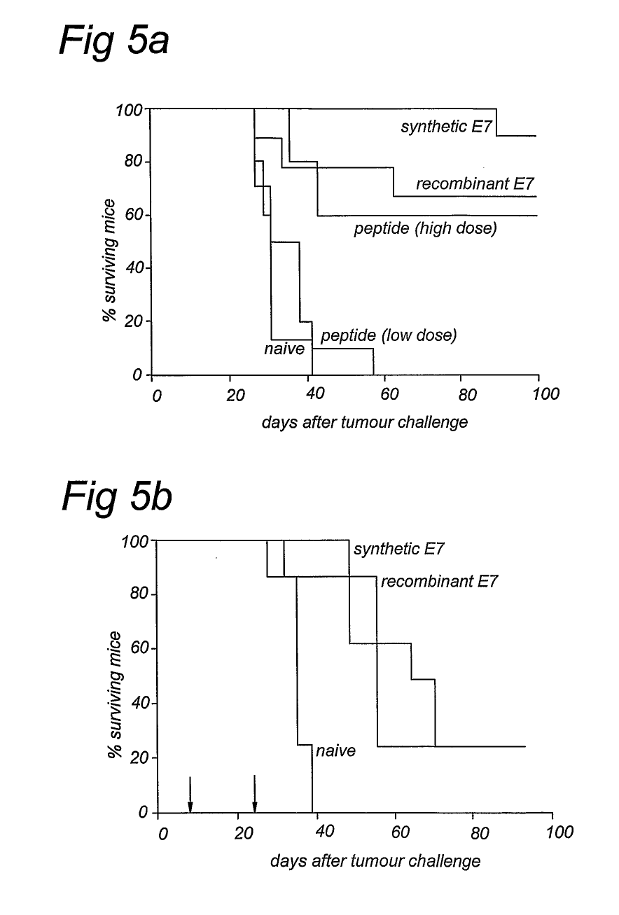

Following a challenge with 50,000 TC-1 tumour cells, naïve mice started to develop palpable tumours from day 11 onwards and all mice died within 41 days after tumour inoculation. Mice vaccinated with 4.5 nmol of the synthetic E7 protein in a prime-boost protocol were protected till day 70. After this day only one of the ten mice vaccinated with synthetic E7 developed a tumour while from the group of mice that were vaccinated with recombinant E7 protein 3 mice died. When mice were vaccinated with an equimolar amount of long E7 peptide all mice developed a tumour (FIG. 5a) but of the mice injected with the previously established protective, approximately 8-fold, higher dose of long peptide (13) only 4 developed a tumour. In order to test the capacity of this vaccine to eradicate an established tumour, mice were first challenged with TC-l tumour cells and vaccinated at day 9 when tumours were palpable. A booster vaccination was given 14 days later. Naive mice all died within 39 days after tumour challenge. Of the mice injected with a low dose of long peptide (equimolar amount to synthetic protein) >40% were still alive at day 40 but eventually all mice died before day 56. Of the 8 mice that were vaccinated with synthetic E7 protein six mice died between day 50 and 70, while the last 2 mice survived at least until to day 95. Mice vaccinated with recombinant E7 protein died somewhat quicker but the survival kinetics were similar to that of the group vaccinated with the synthetic protein (FIG. 5b).

The experiments above demonstrate that the synthetic E7 protein induces strong CD8+ IFNγ-producing T-cell immunity that is capable of protecting mice against tumours in both prophylactic and therapeutic settings. Therapeutic vaccination with synthetic E7 did not result in the eradication of tumours in all mice but this may be related to the aggresiveness of TC-1 tumour growth observed in these experiments. Palpable tumours not only appeared significantly earlier after injection of TC-1 cells but also their growth rate was higher since all mice died within 7days (FIG. 5b) while in our previous experiments naive mice died within 14-21 days (Van der Burg S. H. Vaccine 19, p3652-60, 2001). Even the high-dose of long peptide, which in our previous study protected mice extremely well (Zwaveling S. et al., J. Immunol. 169, p350-8, 2002), was not capable of completely preventing the development of tumours in our current study.

Compared with the recombinant or the synthetic E7 protein, vaccination with an equimolar dose of long E7 peptide was not efficient in the induction of CD8+ IFNγ-producing HPV16 E7-specific T-cells and this was reflected in the lack of protection against a tumour challenge. Since vaccination with a 8 fold higher dose of long peptide did result in a strong E7-specific T-cell response and tumour protection and that the longevity of antigen presentation at the site of CpG mediated DC activation plays an important role. This is related to the in vitro/vivo susceptibility of peptides and proteins to proteolytic breakdown and with differences in the spreading of proteins and peptides, but also of the adjuvant CpG, throughout the body. Our data show that at an equimolar basis, synthetic proteins have a more favourable profile for vaccination purposes.

1-22. (canceled)

23. A method for producing a synthetic protein comprising an amino acid sequence that is at least 80% identical to at least 46 contiguous amino acids of a naturally occurring antigenic protein of a pathogen or tumor, comprising the steps of:
a) chemically synthesizing two or more fragments each consisting of 2-80 contiguous amino acids of the amino acid sequence, wherein the two or more fragments in the amino acid sequence are neighbouring and non-overlapping;
b) chemically ligating the C-terminus of a fragment to the N-terminus of a neighbouring fragment to produce the synthetic protein or a part thereof; and
c) optionally, repeating step b) to sequentially ligate a further neighbouring fragment obtained from step b) or step c) to produce the synthetic protein.

24. The method according to claim 23, wherein the neighbouring non-overlapping fragments comprise a N-terminal cysteine or a glycine residue.

25. The method according to claim 23, wherein the naturally occurring protein is an HPV protein.

26. The method according to claim 25, wherein the HPV protein is an E2, E6 or E7 protein from HPV16, HPV18, HPV31, HPV33 or HPV45.

27. The method according to claim 23, wherein the synthetic protein obtained from step b) or c) is chemically conjugated to an adjuvant.

28. The method according to claim 27, wherein the adjuvant is chemically synthesized.

29. The method according to claim 27, wherein the adjuvant is capable of activating dendritic cells.

30. The method according to claim 29, wherein the adjuvant is selected from the group consisting of polyIC, CpG DNA, imiquimod, Pam3Cys, LPS and combinations thereof.

31. The method according to claim 23, further comprising the step of formulating the synthetic protein into a pharmaceutical composition by mixing the protein with a pharmaceutically acceptable carrier.

32. A composition comprising a synthetic protein, the protein comprising an amino acid sequence that is at least 80% identical to at least 46 contiguous amino acids of a naturally occurring antigenic protein of a pathogen or tumor, wherein the composition is free of a nucleic acid encoding the amino acid sequence.

33. The composition according to claim 32, wherein the protein comprises an amino acid sequence that is at least 80% identical to at least 46 contiguous amino acids of one of SEQ ID NOS. 1 to 6, wherein the composition is free of DNA encoding the amino acid sequences of SEQ ID NOS. 1 to 6.

34. The composition according to claim 33, wherein the composition further comprises an adjuvant.

35. The composition according to claim 34, wherein the adjuvant is capable of activating dendritic cells.

36. The composition according to claim 35, wherein the adjuvant is a TLR-activating adjuvant selected from the group consisting of polyIC, CpG DNA, imiquimod, Pam3Cys, LPS and combinations thereof.

37. The composition according to claim 34, wherein the adjuvant is covalently conjugated to the protein.

38. The composition according to claim 31, wherein the composition further comprises a pharmaceutically acceptable carrier.

39. The composition according to claim 32, wherein the composition further comprises anti-CD40 antibody.

40. The composition according to claim 39, for use as a vaccine.

41. A method for the treatment or prevention of an HPV-associated disease, comprising administering to a subject a synthetic protein produced as defined in claim 23, or a composition as defined in claim 32, in a therapeutically effective amount.

42. The method according to claim 41, wherein the HPV-associated disease is an HPV-induced cancer.

43. A method for producing a medicament for the prevention or treatment of an HPV-associated disease, comprising manufacturing a synthetic protein produced as defined in claim 23, or a composition as defined in claim 32.

44. The method according to claim 43, wherein the HPV-associated disease is an HPV-induced cancer.
